(19) United States
(12) Patent Application Publication
(10) Pub. No.: US 2026/0242433 A1
(43) Pub. Date: Aug. 20, 2026

(54) Tmem52 PROTEIN FRAGMENT, ENCODING GENE, RECOMBINANT VECTOR, RECOMBINANT MICROORGANISM, AND USE THEREOF

(71) Applicant: Peking University, Beijing (CN)

(72) Inventors: Ruimao ZHENG, Beijing (CN); Jiarui LIU, Beijing (CN)

(21) Appl. No.: 18/843,796

(22) PCT Filed: Sep. 5, 2023
(86) PCT No.: PCT/CN2023/116906

§ 371 Date: Sep. 4, 2024

(30) Foreign Application Priority Data
Apr. 27, 2023 (CN) 202310465568.X

(51) Int. Cl. C07K 14/47; A61K 9/00; A61K 38/00; A61P 3/06; A61P 3/10

(52) U.S. Cl. CPC C07K 14/4702; A61K 9/0019; A61P 3/06; A61P 3/10; A61K 38/00

(57) ABSTRACT

A Tmem52 protein fragment, an encoding gene, a recombinant vector, a recombinant microorganism, and use thereof are provided, belonging to the technical field of biomedicine. The Tmem52 protein fragment can reduce body weight and increase basal metabolic rate, thereby preventing and treating obesity; the protein fragment can enhance insulin sensitivity and improve blood glucose regulation ability, thus preventing and treating diabetes; the protein fragment can also reduce serum triglyceride levels, thereby preventing and treating hyperlipidemia. The Tmem52 protein fragment can be used for the treatment of obesity and related diseases thereof, such as diabetes and hyperlipidemia.

### CROSS REFERENCE TO RELATED APPLICATION

The present application is a national stage application of International Patent Application No. PCT/CN2023/116906, filed on Sep. 5, 2023, which claims priority to Chinese Patent Application No. 202310465568.X filed to the China National Intellectual Property Administration (CNIPA) on Apr. 27, 2023 and entitled “Tmem52 PROTEIN FRAGMENT, ENCODING GENE, RECOMBINANT VECTOR, RECOMBINANT MICROORGANISM, AND USE THEREOF”, which is incorporated herein by reference in its entirety.

### REFERENCE TO SEQUENCE LISTING

A computer readable XML file entitled “Sequence Listing”, that was created on Aug. 19, 2024, with a file size of 5,643 bytes, contains the sequence listing for this application, has been filed with this application, and is hereby incorporated by reference in its entirety.

### TECHNICAL FIELD

The present disclosure belongs to the technical field of biomedicine, and specifically relates to a Tmem52 protein fragment, an encoding gene, a recombinant vector, a recombinant microorganism, and use thereof.

### BACKGROUND

Obesity is defined as excessive accumulation and/or abnormal distribution of body fat and body weight gain. It is a complex, multifactorial, chronic metabolic disease caused by a variety of factors including genetics and the environment. As an important risk factor for the onset of non-communicable diseases, obesity was listed by the World Health Organization (WHO) in 2013 as the third leading risk factor for mortality worldwide, following hypertension and smoking. Obesity can lead to lipid metabolism disorders, causing hyperlipidemia and atherosclerotic diseases; obesity can increase the heart load and raise blood pressure, thereby causing cardiovascular diseases; obesity can cause insulin resistance and relative or absolute insulin deficiency, leading to type 2 diabetes; the obesity can lead to liver fat hydrolysis disorders and liver fatty deposition, resulting in non-alcoholic fatty liver disease (NAFLD). In addition, obesity can also cause cerebrovascular diseases, cancer, sexual dysfunction, and abnormal growth and development of adolescents, and so on. Obesity leads to a sharp decline in the quality of life and life expectancy of the people.

Existing patent CN114213518A discloses a Tmem52 gene that regulates glucose and lipid metabolism, and knocking out the Tmem52 gene can promote abnormal glucose and lipid metabolism.

However, there are currently no reports on use of a Tmem52 protein fragment for the prevention and/or treatment of obesity and related diseases thereof (such as diabetes and hyperlipidemia).

### SUMMARY

In view of this, the purpose of the present disclosure is to provide a Tmem52 protein fragment, an encoding gene, a recombinant vector, a recombinant microorganism, and use thereof. In the present disclosure, the Tmem52 protein fragment can be used to prepare a drug for preventing and/or treating obesity and related diseases thereof (such as diabetes and hyperlipidemia).

The present disclosure provides a Tmem52 protein fragment, where the Tmem52 protein fragment is one selected from the group consisting of polypeptides defined in (1) and (2):
(1) a polypeptide with an amino acid sequence set forth in SEQ ID NO: 1; and
(2) a polypeptide derived from the polypeptide defined in (1) by substituting, deleting, or adding one or more amino acids in the amino acid sequence of the polypeptide defined in (1), by chemically modifying one or more amino acid residues in the amino acid sequence of the polypeptide defined in (1), or by cyclizing the amino acid sequence of the polypeptide defined in (1), and has a same function as the polypeptide defined in (1).

The present disclosure further provides an encoding gene of the Tmem52 protein fragment.

In some embodiments, a cDNA nucleotide sequence of the encoding gene is set forth in SEQ ID NO: 2.

The present disclosure further provides a recombinant vector inserted with the encoding gene.

The present disclosure further provides a recombinant microorganism with the recombinant vector.

The present disclosure further provides use of the Tmem52 protein fragment, the encoding gene, the recombinant vector, or the recombinant microorganism in one or more of 1) to 7):
1) preparation of a drug for preventing and/or treating obesity;
2) preparation of a drug for preventing and/or treating diabetes;
3) preparation of a drug for preventing and/or treating hyperlipidemia;
4) preparation of a drug for preventing and/or treating other obesity-related diseases; where the obesity-related diseases include but are not limited to hypertension, fatty liver, atherosclerotic diseases, cardiovascular diseases, cerebrovascular diseases, metabolic syndrome, cancer, and abnormal growth and development;
5) preparation of a drug for increasing basal metabolic rate and/or reducing body weight;
6) preparation of a drug for enhancing insulin sensitivity and improving blood glucose regulation ability; and
7) preparation of a drug for improving blood lipid regulation ability.

The present disclosure further provides use of the Tmem52 protein fragment, the encoding gene, the recombinant vector, or the recombinant microorganism in one or more of 8) to 14):
8) prevention and/or treatment of obesity;
9) prevention and/or treatment of diabetes;
10) prevention and/or treatment of hyperlipidemia;
11) prevention and/or treatment of other obesity-related diseases; where the obesity-related diseases are one or more selected from the group consisting of hypertension, fatty liver, atherosclerotic diseases, cardiovascular diseases, cerebrovascular diseases, metabolic syndrome, cancer, and abnormal growth and development;
12) increase of basal metabolic rate and/or reduction of body weight;
13) enhancement of insulin sensitivity and improvement of blood glucose regulation ability; and
14) improvement of blood lipid regulation ability.

In the present disclosure, C57BL6/n mice fed with high-fat diet are used as research subjects. The preparation of the Tmem52 protein fragment provided by the present disclosure can reduce body weight and increase basal metabolic rate, thereby preventing and/or treating obesity; the preparation can enhance insulin sensitivity and improve blood glucose regulation ability, thus preventing and/or treating diabetes; the preparation can also reduce serum triglyceride levels, thereby preventing and/or treating hyperlipidemia. It is evident that the Tmem52 protein fragment can regulate abnormal glucose and lipid metabolism to achieve the prevention and/or treatment of obesity and its related diseases.

### BRIEF DESCRIPTION OF THE DRAWINGS

FIG. 1 is a graph showing the changes in body weight of mice in the blank solvent (phosphate-buffered saline, PBS) group and the treatment groups with gradient concentrations of Tmem52 protein fragment preparations, ***P<0.001;

FIG. 2A-2B show the thermal image of body surface temperature of mice in the blank solvent (phosphate-buffered saline, PBS) group and the treatment groups with gradient concentrations of Tmem52 protein fragment preparations; where FIG. 2A shows the thermal image of the body surface temperature of mice after 4 weeks of treatment, while FIG. 2B shows the thermal image of the body surface temperature of mice after 8 weeks of treatment;

FIG. 3 shows the energy expenditure results of mice in the blank solvent (phosphate-buffered saline, PBS) group and the treatment groups with gradient concentrations of Tmem52 protein fragment preparations;

FIG. 4A-4D show the results of glucose tolerance test and insulin tolerance test of mice in the blank solvent (phosphate-buffered saline, PBS) group and the treatment groups with gradient concentrations of Tmem52 protein fragment preparations; where FIG. 4A shows the result of the glucose tolerance test after 4 weeks of treatment; FIG. 4B shows the result of the insulin tolerance test after 4 weeks of treatment; FIG. 4C shows the result of the glucose tolerance test after 8 weeks of treatment; FIG. 4D shows the result of the insulin tolerance test after 8 weeks of treatment; *P<0.05, **P<0.01, ***P<0.001; and

FIG. 5 shows the measurement results of serum triglyceride concentration of mice in the blank solvent (phosphate-buffered saline, PBS) group and the group treated with 0.5 mg/kg/d of Tmem52 protein fragment preparation, *P<0.05.

### DETAILED DESCRIPTION OF THE EMBODIMENTS

The present disclosure provides the Tmem52 protein fragment, where the Tmem52 protein fragment is one selected from the group consisting of polypeptides defined in (1) and (2):
(1) a polypeptide with an amino acid sequence set forth in SEQ ID NO: 1; and
(2) a polypeptide derived from the polypeptide defined in (1) by substituting, deleting, or adding one or more amino acids in the amino acid sequence of the polypeptide defined in (1), by chemically modifying one or more amino acid residues in the amino acid sequence of the polypeptide defined in (1), or by cyclizing the amino acid sequence of the polypeptide defined in (1), and has a same function as the polypeptide defined in (1).

In the present disclosure, the amino acid sequence shown in SEQ ID NO: 1 is specifically:

SCVRFCCLRKQTHTQSHTPAAWQPCDGTVIPVDSDSPAHSTVTSYSSVQY
PLGMRLPLYFGEPDPDSMVPPTYSLYASELPPSYDEVVKMIKAREEVAAP
SEKTNSLPEALEPETTGGPQEPGPSAQRP.

In the present disclosure, the Tmem52 protein fragment can reduce body weight and increase basal metabolic rate, thereby preventing obesity; it can also enhance insulin sensitivity and improve blood glucose regulation ability, thus preventing diabetes. The Tmem52 protein fragment can regulate abnormal glucose and lipid metabolism to achieve the treatment of diabetes and obesity.

In the present disclosure, the amino acid sequence of the Tmem52 protein is set forth in SEQ ID NO: 3, specifically:

MAPGPSATQGILLLLPLLPLSQVTLGSADRNCDPSDQCPPQARWSSLWHV
GLILLAILLMLLCGVTASCVRFCCLRKQTHTQSHTPAAWQPCDGTVIPVD
SDSPAHSTVTSYSSVQYPLGMRLPLYFGEPDPDSMVPPTYSLYASELPPS
YDEVVKMIKAREEVAAPSEKTNSLPEALEPETTGGPQEPGPSAQRP.

The underlined part represents the sequence of the Tmem52 protein fragment.

The present disclosure further provides the encoding gene of the Tmem52 protein fragment.

In the present disclosure, the cDNA of the encoding gene of the Tmem52 protein fragment has a nucleotide sequence set forth in SEQ ID NO: 2, specifically:

agctgtgtacgattctgctgcctcaggaagcagacgcacacccagtcaca
cacgccagcggcgtggcagccctgtgacgggacagtcatcccagtggaca
gtgatagccccgcacacagcactgtgacctcctacagttccgtgcagtac
ccactgggcatgcggctgcccctgtactttggagagccagaccctgactc
catggtccctcccacctacagcctctatgcgtctgagctgccaccctcct
acgatgaagttgtgaagatgataaaagccagggaggaagtggcagctccc
tctgagaaaaccaactctctgcctgaggccttggagccagagaccactgg
agggccccaggagccaggccccagtgcccagcggccctag.

In the present disclosure, Tmem52 gene encoding the full-length Tmem52 protein has a nucleotide sequence set forth in SEQ ID NO: 4, specifically:

atggccccggggccgtcagccacacaggggatcctgctgctgctgcctct
cctgcctctgtcccaggtgacgctgggttccgcggaccgcaactgtgacc
cctcggatcagtgcccgccccaggcccgctggagcagcctatggcatgtg
gggctcatcctgctcgccatactcctgatgcttttgtgtggggtcacagc
cagctgtgtacgattctgctgcctcaggaagcagacgcacacccagtcac
acacgccagcggcgtggcagccctgtgacgggacagtcatcccagtggac
agtgatagccccgcacacagcactgtgacctcctacagttccgtgcagta
cccactgggcatgcggctgcccctgtactttggagagccagaccctgact
ccatggtccctcccacctacagcctctatgcgtctgagctgccaccctcc
tacgatgaagttgtgaagatgataaaagccagggaggaagtggcagctcc
ctctgagaaaaccaactctctgcctgaggccttggagccagagaccactg
gagggccccaggagccaggccccagtgcccagcggccctag.

The present disclosure further provides the recombinant vector inserted with the encoding gene of the Tmem52 protein fragment.

The present disclosure further provides the recombinant microorganism with the recombinant vector inserted with the encoding gene of the Tmem52 protein fragment; where the original bacterium of the recombinant microorganism is preferably Escherichia coli.

In the present disclosure, there is no particular limitation on the method for constructing the recombinant plasmid, recombinant microorganism, or an animal that overexpresses Tmem52 protein fragment, and conventional methods in the art may be used.

The present disclosure further provides use of the Tmem52 protein fragment, the encoding gene, the recombinant vector, or the recombinant microorganism in one or more of 1) to 7):
1) preparation of a drug for preventing and/or treating obesity;
2) preparation of a drug for preventing and/or treating diabetes;
3) preparation of a drug for preventing and/or treating hyperlipidemia;
4) preparation of a drug for preventing and/or treating other obesity-related diseases; where the obesity-related diseases include but are not limited to hypertension, fatty liver, atherosclerotic diseases, cardiovascular diseases, cerebrovascular diseases, metabolic syndrome, cancer, and abnormal growth and development;
5) preparation of a drug for increasing basal metabolic rate and/or reducing body weight;
6) preparation of a drug for enhancing insulin sensitivity and improving blood glucose regulation ability; and
7) preparation of a drug for improving a blood lipid regulation ability.

In the specific implementation of the present disclosure, the Tmem52 protein fragment is dissolved in PBS for administration; for mice, the Tmem52 protein fragment is preferably administered at the concentration of 0.001 to 1 mg/mL; the Tmem52 protein fragment is preferably administered by intraperitoneal injection; for mice, the Tmem52 protein fragment is preferably injected at the dosage of 0.005 to 5 mg/kg/d.

The present disclosure further provides use of the Tmem52 protein fragment, the encoding gene, the recombinant vector, or the recombinant microorganism in one or more of 8) to 14):
8) prevention and/or treatment of obesity;
9) prevention and/or treatment of diabetes;
10) prevention and/or treatment of hyperlipidemia;
11) prevention and/or treatment of other obesity-related diseases; where the obesity-related diseases are one or more selected from the group consisting of hypertension, fatty liver, atherosclerotic diseases, cardiovascular diseases, cerebrovascular diseases, metabolic syndrome, cancer, and abnormal growth and development;
12) increase of basal metabolic rate and/or reduction of body weight;
13) enhancement of insulin sensitivity and improvement of blood glucose regulation ability; and
14) improvement of blood lipid regulation ability.

In the present disclosure, an injection primarily composed of the Tmem52 protein fragment is administered in a unit dose form; the dosage of the Tmem52 protein fragment in the injection is 0.005 mg/kg to 5 mg/kg of the Tmem52 protein fragment per day; and the concentration of the Tmem52 protein fragment in the injection is preferably 0.001 mg/mL to 1 mg/mL.

The present disclosure further provides drugs or health care products, including the Tmem52 protein fragments and pharmaceutically acceptable excipients.

In the present disclosure, the excipients include PBS; and the dosage form of the drug or the health care product includes injections.

The technical solutions of the present disclosure will be clearly and completely described below with reference to the examples of the present disclosure.

### Example 1

Cloning and purification of the Tmem52 protein fragment, which included the following steps:
1) A target DNA fragment corresponding to the Tmem52 protein fragment was obtained (primers were designed based on the sequence set forth in SEQ ID NO: 2 for amplifying the DNA fragment).
2) The recombinant plasmid was constructed.
3) The recombinant plasmid was introduced into Escherichia coli (E. coli) for expression, purification, and identification, including:
100 μL of competent E. coli BL21 (DE3) cells were taken and added with 4 μL of the recombinant plasmid constructed in step 2), the obtained mixture were incubated on ice for 30 min, heated shock at 42° C. for 45 s, and incubated on ice for 1 min; 700 μL of LB medium was added to the mixture incubated on ice, shaken at 37° C. for 45 min; the result cells were grown overnight on a plate; single colonies were transferred in 50 mL of LB liquid medium and shaken overnight; 10 mL of bacterial solution was inoculated in a large bottle containing 1 L of LB medium, shaken until the OD value was 0.6 to 0.8; 1 mL of IPTG (working conc. 1 mM) was added to each 1 L of bacterial solution, induced at 25° C. for 8 h; the bacterial solution was centrifuged at 5,000 rpm for 25 min, the supernatant was removed, the bacterial cells were resuspended with 25 mL of 8 wt. % NaCl solution, transferred to a 50 mL corning tube, centrifuged (at 5,000 rpm for 25 min), the supernatant was removed, and the bacterial sludge was stored at −80° C.

The bacterial sludge was resuspended in 40 mL lysis buffer and lysed by ultrasonication on ice for 3 rounds, each round for 13 min; after ultrasonication, centrifugation was conducted at 12,000 rpm for 15 min at 4° C. to obtain a preliminarily purified mouse Tmem52 protein with a his tag; further purification was conducted using a Ni affinity column, followed by elution, and gel electrophoresis was conducted after elution to determine the purity of the protein; after purification, the his tag was removed.
4) Endotoxin was removed by agarose gel affinity chromatography to obtain the Tmem52 protein fragment suitable for in vivo injection.

### Example 2

40 wild-type mice used in this experiment were 8-week-old male, healthy SPF-grade C57BL6/n mice purchased from the Animal Center of Peking University Health Science Center. The mice were fed at a controlled environmental temperature of 22±0.5° C., with a 12 h/12 h light/dark cycle. The following experimental data were represented as the mean±standard deviation of the mice within the same group.

After 7 days of adaptive training on ordinary feed (purchased from Jiangsu Xietong Pharmaceutical Bio-engineering Co., Ltd., catalog number SZS9126), the mice were fed with high-fat feed (purchased from Research diets, catalog number D12492i) for 8 weeks and divided into 5 groups, with 8 mice in each group. During the high-fat diet feeding, the 5 groups of mice were intraperitoneally injected with blank solvent (PBS) and Tmem52 protein fragment preparations of gradient concentrations (prepared in Example 1), with specific dosages of 0.005 mg/kg/d, 0.05 mg/kg/d, 0.5 mg/kg/d, and 5 mg/kg/d, respectively. The solvent of the Tmem52 protein fragment preparation was PBS.

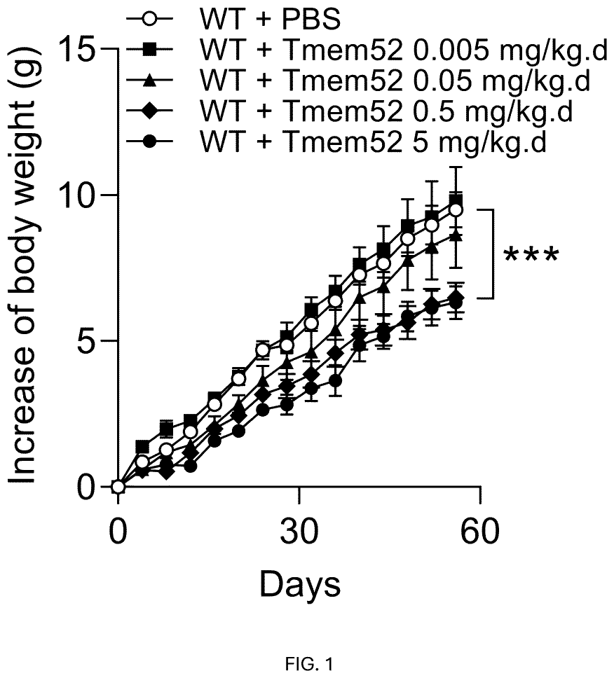

During the experiment, starting from the first day of the first week of drug administration, the body weight of mice was measured every 4 days, and the body weight change results of the mice were obtained by subtracting the initial body weight of the mice from the weighed body weight, lasting for 8 weeks. Experimental results were shown in Table 1 and FIG. 1.

TABLE 1
Calculation results of body weight changes of mice in different groups (unit: g)

| | | Tmem52 | Tmem52 | Tmem52 | Tmem52 |
|---|---|---|---|---|---|
| Time | | protein fragment | protein fragment | protein fragment | protein fragment |
| (day) | PBS | 0.005 mg/kg/d | 0.05 mg/kg/d | 0.5 mg/kg/d | 5 mg/kg/d |
| 0 | 0.0 | 0.0 | 0.0 | 0.0 | 0.0 |
| 4 | 0.9 | 1.4 | 0.6 | 0.6 | 0.6 |
| 8 | 1.3 | 2.0 | 1.2 | 0.5 | 0.8 |
| 12 | 1.9 | 2.3 | 1.4 | 1.2 | 0.7 |
| 16 | 2.8 | 3.0 | 2.1 | 2.0 | 1.6 |
| 20 | 3.7 | 3.8 | 2.8 | 2.4 | 1.9 |
| 24 | 4.7 | 4.7 | 3.7 | 3.2 | 2.6 |
| 28 | 4.9 | 5.1 | 4.3 | 3.5 | 2.8 |
| 32 | 5.6 | 6.1 | 4.6 | 3.9 | 3.4 |
| 36 | 6.4 | 6.7 | 5.4 | 4.6 | 3.6 |
| 40 | 7.3 | 7.6 | 6.5 | 5.2 | 4.9 |
| 44 | 7.7 | 8.2 | 6.9 | 5.4 | 5.2 |
| 48 | 8.5 | 8.9 | 7.8 | 5.6 | 5.8 |
| 52 | 9.0 | 9.2 | 8.3 | 6.3 | 6.1 |
| 56 | 9.5 | 9.8 | 8.6 | 6.5 | 6.3 |

As shown in Table 1 and FIG. 1, after 8 weeks of treatment with Tmem52 protein fragment preparations at doses of 0.5 mg/kg/d and 5 mg/kg/d, the body weight gain of mice was significantly lower than that of the control group. After 8 weeks of treatment with Tmem52 protein fragment preparations at doses of 0.05 mg/kg/d and 0.005 mg/kg/d, the body weight gain of mice was not significantly different from that of the control group. It was evident that the Tmem52 protein fragment preparation had a weight loss effect, and the Tmem52 protein fragment preparation and its composition might be used to treat obesity and/or obesity-related diseases.

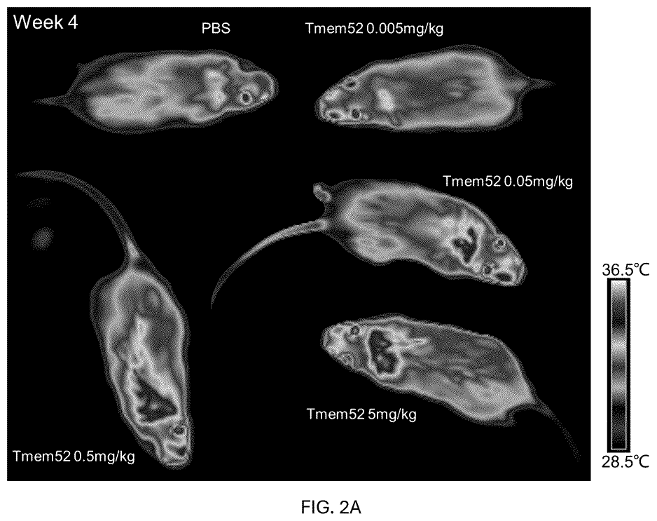

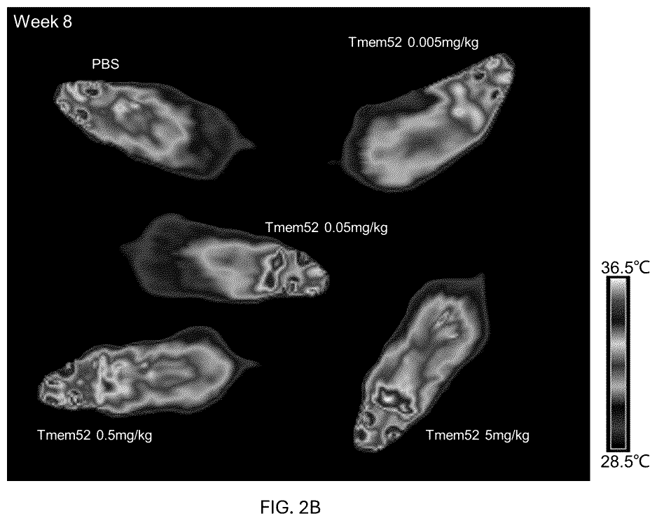

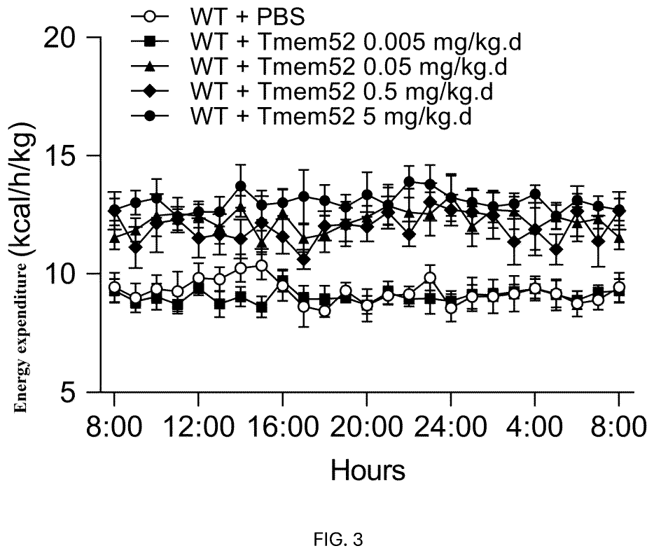

The epidermal body temperature of mice was measured after 4 and 8 weeks of treatment with the Tmem52 protein fragment. The results of the measurements were shown in FIG. 2A-2B. After 8 weeks of treatment with the Tmem52 protein fragment, the energy expenditure of mice was monitored using a respiratory metabolic analysis system (6 mice per group). Test results were shown in Table 2 and FIG. 3.

TABLE 2
Energy expenditure results of mice in different groups (unit: kJ/h/kg)

| | | Tmem52 | Tmem52 | Tmem52 | Tmem52 |
|---|---|---|---|---|---|
| | | protein fragment | protein fragment | protein fragment | protein fragment |
| Time | PBS | 0.005 mg/kg/d | 0.05 mg/kg/d | 0.5 mg/kg/d | 5 mg/kg/d |
| 8:00 | 9.428 | 9.287 | 11.543 | 12.671 | 12.723 |
| 9:00 | 8.980 | 8.827 | 11.861 | 11.116 | 13.017 |
| 10:00 | 9.354 | 9.027 | 12.466 | 12.146 | 13.200 |
| 11:00 | 9.259 | 8.711 | 12.544 | 12.302 | 12.433 |
| 12:00 | 9.820 | 9.378 | 12.452 | 11.521 | 12.625 |
| 13:00 | 9.771 | 8.727 | 11.992 | 11.671 | 12.635 |
| 14:00 | 10.233 | 9.026 | 12.860 | 11.476 | 13.713 |
| 15:00 | 10.342 | 8.598 | 11.327 | 12.168 | 12.914 |
| 16:00 | 9.497 | 9.710 | 12.596 | 11.582 | 13.014 |
| 17:00 | 8.623 | 8.927 | 11.504 | 10.606 | 13.277 |
| 18:00 | 8.436 | 8.941 | 11.684 | 12.035 | 13.110 |
| 19:00 | 9.280 | 8.984 | 12.110 | 12.089 | 12.830 |
| 20:00 | 8.666 | 8.684 | 12.410 | 11.999 | 13.358 |
| 21:00 | 9.083 | 9.202 | 12.876 | 12.605 | 12.921 |
| 22:00 | 9.125 | 8.932 | 12.604 | 11.687 | 13.901 |
| 23:00 | 9.826 | 8.959 | 12.537 | 13.045 | 13.798 |
| 24:00 | 8.562 | 8.850 | 13.306 | 12.704 | 13.228 |
| 1:00 | 9.026 | 9.132 | 12.005 | 12.621 | 13.036 |
| 2:00 | 9.046 | 9.107 | 12.723 | 12.469 | 12.853 |
| 3:00 | 9.156 | 9.248 | 12.658 | 11.358 | 12.964 |
| 4:00 | 9.378 | 9.366 | 11.896 | 11.885 | 13.380 |
| 5:00 | 9.151 | 9.115 | 12.414 | 11.034 | 12.459 |
| 6:00 | 8.739 | 8.885 | 12.163 | 12.657 | 13.114 |
| 7:00 | 8.890 | 9.225 | 12.336 | 11.384 | 12.866 |
| 8:00 | 9.428 | 9.287 | 11.543 | 12.671 | 12.723 |

As shown in FIG. 2A-2B, atter 4 weeks of treatment with Tmem52 protein fragment preparations at doses of 0.05 mg/kg/d, 0.5 mg/kg/d, and 5 mg/kg/d, the epidermal body temperature of mice was significantly higher than that of the control group; after 8 weeks of treatment with Tmem52 protein fragment preparations at doses of 0.05 mg/kg/d, 0.5 mg/kg/d, and 5 mg/kg/d, the epidermal body temperature of mice was still significantly higher than that of the control group. As shown in Table 2 and FIG. 3, after 8 weeks of treatment with Tmem52 protein fragment preparations at doses of 0.05 mg/kg/d, 0.5 mg/kg/d, and 5 mg/kg/d, the energy expenditure of mice was significantly higher than that of the control group.

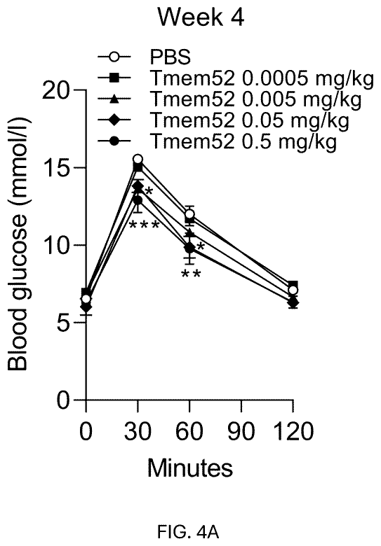

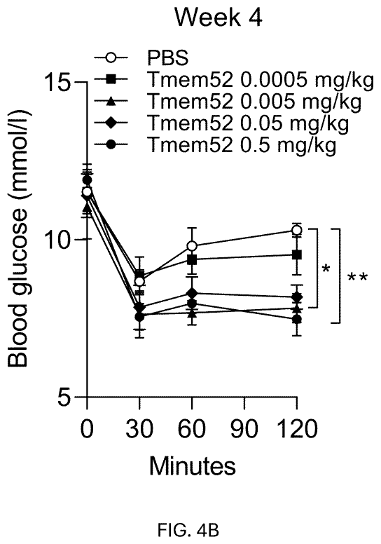

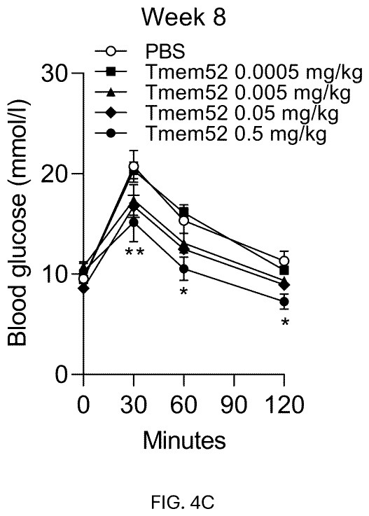

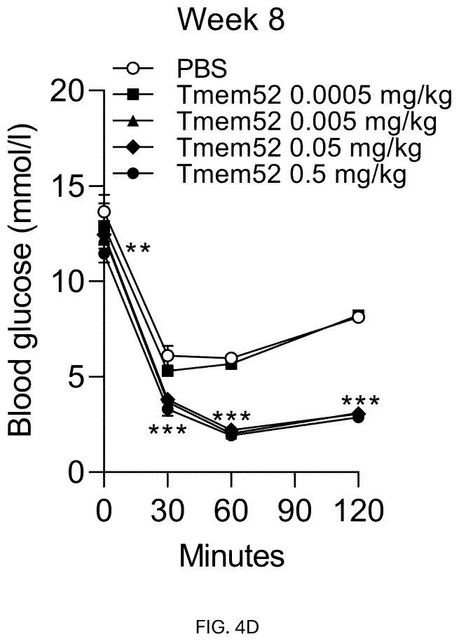

After 4 and 8 weeks of treatment with the Tmem52 protein fragment, glucose tolerance test and insulin tolerance test were conducted separately (4 mice per group). The procedure for the glucose tolerance test was as follows: after the mice fasted for 16 h, glucose (2 g/kg) was injected intraperitoneally, and blood was collected from the tip of the tail to measure blood glucose levels (four time points in total: before glucose injection, and 30, 60, and 120 min after glucose injection). The procedure for the insulin tolerance test was as follows: after the mice fasted for 4 h, insulin (0.5 U/kg) was injected intraperitoneally, and blood was collected from the tip of the tail to measure blood glucose levels (four time points in total: before insulin injection, and 30, 60, and 120 min after insulin injection). Test results were shown in Table 3, Table 4, Table 5, Table 6, and FIG. 4A-4D.

TABLE 3
Results of glucose tolerance test of mice in different
groups after 4 weeks of treatment (unit: mmol/L)

| | | Tmem52 | Tmem52 | Tmem52 | Tmem52 |
|---|---|---|---|---|---|
| Time | | protein fragment | protein fragment | protein fragment | protein fragment |
| (min) | PBS | 0.005 mg/kg/d | 0.05 mg/kg/d | 0.5 mg/kg/d | 5 mg/kg/d |
| 0 | 6.5 | 6.9 | 6.8 | 6.0 | 6.3 |
| 30 | 15.5 | 15.0 | 13.6 | 13.8 | 12.9 |
| 60 | 12.0 | 11.7 | 10.8 | 9.9 | 9.8 |
| 120 | 7.1 | 7.4 | 6.7 | 6.3 | 6.3 |

TABLE 4
Results of insulin tolerance test of mice in different
groups after 4 weeks of treatment (unit: mmol/L)

| | | Tmem52 | Tmem52 | Tmem52 | Tmem52 |
|---|---|---|---|---|---|
| Time | | protein fragment | protein fragment | protein fragment | protein fragment |
| (min) | PBS | 0.005 mg/kg/d | 0.05 mg/kg/d | 0.5 mg/kg/d | 5 mg/kg/d |
| 0 | 11.5 | 11.5 | 11.1 | 11.4 | 11.9 |
| 30 | 8.7 | 8.9 | 7.6 | 7.9 | 7.6 |
| 60 | 9.8 | 9.4 | 7.7 | 8.3 | 8.0 |
| 120 | 10.3 | 9.5 | 7.8 | 8.2 | 7.5 |

TABLE 5
Results of glucose tolerance test of mice in different
groups after 8 weeks of treatment (unit: mmol/L)

| | | Tmem52 | Tmem52 | Tmem52 | Tmem52 |
|---|---|---|---|---|---|
| Time | | protein fragment | protein fragment | protein fragment | protein fragment |
| (min) | PBS | 0.005 mg/kg/d | 0.05 mg/kg/d | 0.5 mg/kg/d | 5 mg/kg/d |
| 0 | 9.5 | 9.5 | 10.8 | 8.6 | 10.3 |
| 30 | 20.7 | 20.3 | 17.4 | 16.8 | 15.2 |
| 60 | 15.3 | 16.1 | 13.1 | 12.5 | 10.5 |
| 120 | 11.3 | 10.4 | 9.4 | 8.9 | 7.3 |

TABLE 6
Results of insulin tolerance test of mice in different
groups atter 8 weeks of treatment (unit: mmol/L)

| | | Tmem52 | Tmem52 | Tmem52 | Tmem52 |
|---|---|---|---|---|---|
| Time | | protein fragment | protein fragment | protein fragment | protein fragment |
| (min) | PBS | 0.005 mg/kg/d | 0.05 mg/kg/d | 0.5 mg/kg/d | 5 mg/kg/d |
| 0 | 13.7 | 12.9 | 12.3 | 12.5 | 11.5 |
| 30 | 6.1 | 5.3 | 3.7 | 3.8 | 3.3 |
| 60 | 6.0 | 5.7 | 2.0 | 2.2 | 1.9 |
| 120 | 8.1 | 8.2 | 3.1 | 3.1 | 2.9 |

As shown in Tables 3 to 6 and FIG. 4A-4D, after 4 weeks of treatment with Tmem52 protein preparations at doses of 0.05 mg/kg/d, 0.5 mg/kg/d, and 5 mg/kg/d, the blood glucose regulation ability and insulin sensitivity of mice were higher than those of the control group; after 8 weeks of treatment with Tmem52 protein preparations at doses of 0.05 mg/kg/d, 0.5 mg/kg/d, and 5 mg/kg/d, the blood glucose regulation ability and insulin sensitivity of mice were significantly higher than those of the control group. This suggested that the Tmem52 protein fragment could prevent and treat diabetes.

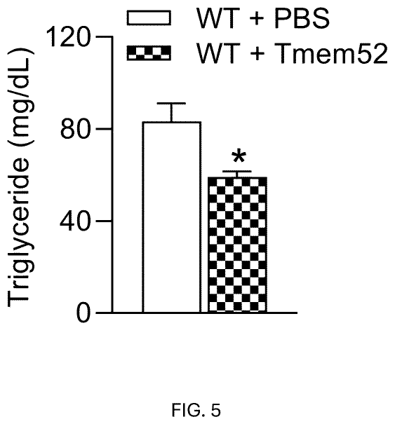

After 8 weeks of treatment with PBS and 0.5 mg/kg/d of Tmem52 protein fragment, serum triglyceride levels were detected (6 mice per group). Test results were shown in Table 7 and FIG. 5.

TABLE 7
Serum triglyceride levels in mice in different
groups after 8 weeks of treatment (unit: mg/dL)

| | PBS | Tmem52 protein fragment 0.5 mg/kg/d |
|---|---|---|
| Serum triglyceride level | 83.36 | 59.19 |

As shown in Table 7 and FIG. 5, after 8 weeks of treatment with 0.5 mg/kg/d of Tmem52 protein preparation, the serum triglyceride level of mice decreased significantly, indicating that the Tmem52 protein treatment could prevent and treat hyperlipidemia.

In summary, the preparation of the Tmem52 protein fragment can reduce body weight and increase basal metabolic rate, thereby preventing and/or treating obesity; the preparation of the protein fragment can enhance insulin sensitivity and improve blood glucose regulation ability, thus preventing and/or treating diabetes; the preparation of the protein fragment can also reduce serum triglyceride levels, thereby preventing and/or treating hyperlipidemia. It can be seen that the Tmem52 protein fragment can regulate abnormal glucose and lipid metabolism to achieve the prevention and/or treatment of obesity and its related diseases.

Although the above example has described the present disclosure in detail, they are only a part of, not all of, the examples of the present disclosure. Other examples may also be obtained by persons based on the examples without creative efforts, and all of these examples shall fall within the protection scope of the present disclosure.

1-10. (canceled)

11. A Tmem52 protein fragment, wherein the Tmem52 protein fragment is one selected from the group consisting of polypeptides defined in (1) and (2):
(1) a polypeptide with an amino acid sequence set forth in SEQ ID NO: 1; and
(2) a polypeptide derived from the polypeptide defined in (1) by substituting, deleting, or adding one or more amino acids in the amino acid sequence of the polypeptide defined in (1), by chemically modifying one or more amino acid residues in the amino acid sequence of the polypeptide defined in (1), or by cyclizing the amino acid sequence of the polypeptide defined in (1), and has a same function as the polypeptide defined in (1).

12. An encoding gene of the Tmem52 protein fragment according to claim 11.

13. The encoding gene according to claim 12, wherein a cDNA nucleotide sequence of the encoding gene is set forth in SEQ ID NO: 2.

14. A recombinant vector inserted with the encoding gene according to claim 12.

15. A recombinant microorganism with the recombinant vector according to claim 14.

16. A method for preventing and/or treating obesity, diabetes, hyperlipidemia or other obesity-related diseases, comprising administering a pharmaceutical composition comprising the Tmem52 protein fragment according to claim 11 to a subject in need thereof,
wherein the obesity-related diseases are one or more selected from the group consisting of hypertension, fatty liver, atherosclerotic diseases, cardiovascular diseases, cerebrovascular diseases, metabolic syndrome, cancer, and abnormal growth and development.

17. The method according to claim 16, wherein an injection primarily composed of the Tmem52 protein fragment is administered in a unit dose form; a dosage of the Tmem52 protein fragment in the injection is 0.005 mg/kg to 5 mg/kg of the Tmem52 protein fragment per day; and a concentration of the Tmem52 protein fragment in the injection is 0.001 mg/mL to 1 mg/mL.

18. A drug or a health care product, comprising the Tmem52 protein fragment according to claim 11 and pharmaceutically acceptable excipients.

19. The drug or the health care product according to claim 18, wherein a dosage form of the drug or the health care product comprises injections.

20. The recombinant vector according to claim 14, wherein a cDNA nucleotide sequence of the encoding gene is set forth in SEQ ID NO: 2.


SEQUENCE LISTING (4 sequences)

SEQ ID NO: 1 - protein, 129 aa, synthetic construct
SCVRFCCLRKQTHTQSHTPAAWQPCDGTVIPVDSDSPAHSTVTSYSSVQYPLGMRLPLYFGEPDPDSMVPPTYSLYASELPPSYDEVVKMIKAREEVAAPSEKTNSLPEALEPETTGGPQEPGPSAQRP

SEQ ID NO: 2 - DNA, 390 bp, synthetic construct
  source 1..390: mol_type = other DNA
agctgtgtacgattctgctgcctcaggaagcagacgcacacccagtcacacacgccagcggcgtggcagccctgtgacgggacagtcatcccagtggacagtgatagccccgcacacagcactgtgacctcctacagttccgtgcagtacccactgggcatgcggctgcccctgtactttggagagccagaccctgactccatggtccctcccacctacagcctctatgcgtctgagctgccaccctcctacgatgaagttgtgaagatgataaaagccagggaggaagtggcagctccctctgagaaaaccaactctctgcctgaggccttggagccagagaccactggagggccccaggagccaggccccagtgcccagcggccctag

SEQ ID NO: 3 - protein, 196 aa, synthetic construct
MAPGPSATQGILLLLPLLPLSQVTLGSADRNCDPSDQCPPQARWSSLWHVGLILLAILLMLLCGVTASCVRFCCLRKQTHTQSHTPAAWQPCDGTVIPVDSDSPAHSTVTSYSSVQYPLGMRLPLYFGEPDPDSMVPPTYSLYASELPPSYDEVVKMIKAREEVAAPSEKTNSLPEALEPETTGGPQEPGPSAQRP

SEQ ID NO: 4 - DNA, 591 bp, synthetic construct
  source 1..591: mol_type = other DNA
atggccccggggccgtcagccacacaggggatcctgctgctgctgcctctcctgcctctgtcccaggtgacgctgggttccgcggaccgcaactgtgacccctcggatcagtgcccgccccaggcccgctggagcagcctatggcatgtggggctcatcctgctcgccatactcctgatgcttttgtgtggggtcacagccagctgtgtacgattctgctgcctcaggaagcagacgcacacccagtcacacacgccagcggcgtggcagccctgtgacgggacagtcatcccagtggacagtgatagccccgcacacagcactgtgacctcctacagttccgtgcagtacccactgggcatgcggctgcccctgtactttggagagccagaccctgactccatggtccctcccacctacagcctctatgcgtctgagctgccaccctcctacgatgaagttgtgaagatgataaaagccagggaggaagtggcagctccctctgagaaaaccaactctctgcctgaggccttggagccagagaccactggagggccccaggagccaggccccagtgcccagcggccctag